(19) United States
(12) Patent Application Publication
(10) Pub. No.: US 2014/0031951 A1
(43) Pub. Date: Jan. 30, 2014

(54) Two-Way Valve

(71) Applicant: Cook Medical Technologies LLC, Bloomington, IN (US)

(72) Inventors: Kieran Costello, Ballina-Killaloe (IE); John Neilan, Gort (IE); Michael Ryan, Limerick (IE)

(73) Assignee: Cook Medical Technologies LLC, Bloomington, IN (US)

(21) Appl. No.: 13/950,820

(22) Filed: Jul. 25, 2013

Related U.S. Application Data
(60) Provisional application No. 61/676,562, filed on Jul. 27, 2012.
(60) Provisional application No. 61/676,570, filed on Jul. 27, 2012.
(60) Provisional application No. 61/681,472, filed on Aug. 9, 2012.

(51) Int. Cl. A61F 2/24

(52) U.S. Cl. CPC A61F 2/24
USPC 623/23.68

(57) ABSTRACT

A medical device and a method for controlling flow through a bodily lumen are provided. The medical device includes a valve device positioned within the bodily lumen. The valve device includes a movable member movable within the bodily lumen and a magnetic portion operably connected to the movable member to facilitate positioning the movable member in a first configuration. The magnetic portion has a magnetic attraction to another magnetic portion or a portion of the valve device having a charge opposite a charge of the magnetic portion. The valve device is movable from the first configuration substantially closing the bodily lumen in the presence of a first pressure within the bodily lumen to a second configuration in response to a second pressure that is greater than the first pressure. The valve device is open in the second configuration and the movable member is normally positioned in the first configuration.

### RELATED APPLICATIONS

This application claims the benefit of U.S. Provisional Application Nos. 61/676,562, filed Jul. 27, 2012; 61/676,570, filed Jul. 27, 2012 and 61/681,472 filed Aug. 9, 2012, which are incorporated by reference herein in their entirety.

### FIELD OF THE INVENTION

The present invention relates to medical devices and in particular to a valve for regulating fluid flow therethrough.

### BACKGROUND OF THE INVENTION

The lower esophageal sphincter (LES) in healthy individuals allows food to pass into the stomach, but prevents gastric fluids from moving into the esophagus except when the patient vomits. Aspiration is a clinical risk for patients having a malfunctioning LES or for patients having stents placed across the LES. Aspiration occurs when the stomach contents travel from the stomach into the lungs. Aspiration in the lungs can lead to pneumonia or death. Risk of aspiration in patients having a comprised LES increases when the patient is in a prone position.

Anti-reflux esophageal prostheses or stents have been developed to treat tumors or strictures in the vicinity of the LES. Anti-reflux esophageal prosthesis or stent is typically placed in the lower esophagus and through the LES to maintain the patency thereof due to the presence of a cancerous tumor commonly found in the vicinity thereof or to treat benign tumor conditions, such as blockage or strictures.

A problem with an esophageal prosthesis or stent is that fluid from the stomach flows into the mouth of the patient when in a prone position, increasing the risk of aspiration. In an attempt to solve the problem, a number of esophageal prostheses or stents utilize a one-way valve in which only food or fluid from the esophagus flows into the stomach in only an antegrade or forward direction. However, these one-way anti-reflux prostheses or stents present another problem. When the patient wants to belch or vomit, the patient is prevented from doing so, because the one-way valve prevents backward flow in the retrograde direction. Such condition is not only painful to the patient, but can also lead to more complicated medical conditions.

What is needed is a prosthesis that allows food to pass into the stomach and prevents gastric fluids from entering the esophagus, yet allows for vomiting and belching when necessary.

### BRIEF SUMMARY

Accordingly, it is an object of the present invention to provide a device and a method having features that resolve or improve on the above-described drawbacks.

In some aspects, a medical device and a method for controlling flow through a bodily lumen are provided. The medical device includes a valve device positioned within the bodily lumen to control the flow through the lumen. The valve device includes a movable member movable within the bodily lumen and a magnetic portion operably connected to the movable member to facilitate positioning the movable member in a first configuration. The magnetic portion has a magnetic attraction to another magnetic portion or a portion of the valve device having a charge opposite a charge of the magnetic portion. The valve device is movable from the first configuration substantially dosing the bodily lumen in the presence of a first pressure within the bodily lumen to a second configuration in response to a second pressure within the bodily lumen that is greater than the first pressure. The valve device is open in the second configuration for flow therethrough. The movable member normally positioned in the first configuration.

In other aspects, a medical device and a method for controlling flow through a bodily lumen in a first direction and a second direction are provided. The medical device includes a tubular member having a proximal portion, a distal portion and a lumen therethrough and a valve device positioned within the lumen. The valve device includes a membrane having a proximal portion and a distal portion and the membrane is operably connected to the tubular member. At least a portion of the membrane a forming a sealed chamber by sealing the proximal portion of the membrane to the proximal portion of the tubular member and sealing the distal portion of the membrane to the distal portion of the tubular member or by sealing the membrane to itself and forming the chamber between the proximal and distal portions of the membrane. The sealed chamber extends inward into the lumen and substantially closes the lumen in a first configuration at a first pressure within the lumen. The membrane having a second configuration in response to a second pressure that is greater than the first pressure wherein the lumen is open in the second configuration for flow therethrough, The sealed chamber includes a filling substance. The membrane is normally positioned in the first configuration, The tubular member, filling substance or both are deformable in response to the second pressure.

### BRIEF DESCRIPTION OF THE DRAWINGS

FIG. 1 is a sectional view of a magnetic valve in accordance with an embodiment of the present invention;

FIGS. 2A-2D illustrate cross-sectional views of the magnet valve in accordance with embodiments of the present invention;

FIG. 3A is a partial sectional view of an embodiment of the magnetic valve in a dosed configuration in response to a first pressure;

FIG. 3B is a partial sectional view of the magnetic valve of FIG. 3A in an open configuration in response to a second pressure;

FIG. 3C is a partial sectional view of the magnetic valve of FIG. 3A in an open configuration in response to a third pressure;

FIG. 3D is a partial sectional view of an embodiment of a magnetic valve an open configuration in response to a second pressure;

FIG. 3E is a partial sectional view of the magnetic valve of FIG. 3D in an open configuration in response to a third pressure;

FIG. 4A is a partial sectional view of an embodiment of the magnetic valve in a dosed configuration;

FIG. 4B is a partial sectional view of the magnetic valve of FIG. 4A in an open configuration in response to a second pressure;

FIG. 4C is a partial sectional view of the magnetic valve of FIG. 4A in an open configuration in response to a third pressure;

FIG. 4D is a partial second view of an alternative embodiment of the magnetic valve in a dosed configuration;

FIG. 5A is a sectional view of an embodiment of the magnetic valve in valve in a dosed configuration;

FIG. 5B is a sectional view of the embodiment of the magnetic valve of FIG. 5A in an open configuration;

FIG. 5C is a sectional view of an embodiment of the magnetic valve;

FIG. 6 is a sectional view of an embodiment of the magnetic valve;

FIG. 7A is a perspective view of an embodiment of the magnetic valve having a sleeve;

FIG. 7B is a cross-section view across line 7-7 of FIG. 7A;

FIG. 7C is a perspective view of the embodiment of the valve of FIG. 7A in an open configuration;

FIG. 7D is an end view of the embodiment shown in FIG. 70;

FIG. 8A is a perspective view of an embodiment of the magnetic valve having a sleeve;

FIG. 8B is sectional view of the embodiment of the magnetic valve shown FIG. 8A with the valve in the open configuration;

FIG. 9 is a perspective view of an embodiment of the magnetic valve having a sleeve;

FIG. 10 is a side view of an embodiment of the magnetic valve having a stent and a sleeve;

FIG. 11 is a side view of an embodiment of the magnetic valve having a stent and a sleeve;

FIG. 12 is a sectional view of an embodiment of a magnetic valve positioned within the lower esophageal sphincter;

FIG. 13 is a sectional view of an embodiment of a prosthesis in accordance with an embodiment of the present invention;

FIG. 14 is a cross sectional view of the prosthesis shown in FIG. 13 in a closed configuration;

FIG. 15 is a cross sectional view of the prosthesis shown in FIG. 13 in an open configuration;

FIG. 16 is a sectional view of an embodiment of a prosthesis in accordance with an embodiment of the present invention;

FIG. 17 is a cross sectional view of the prosthesis shown in FIG. 16 in a closed configuration;

FIG. 18 is a cross sectional view of the prosthesis shown in FIG. 16 in an open configuration;

FIG. 19 is a sectional view of an embodiment of a prosthesis in accordance with an embodiment of the present invention;

FIGS. 20A-20C illustrate cross sectional views of alternative embodiments of the prosthesis shown in FIG. 19 in a closed configuration;

FIG. 21 is a second view of an embodiment of a prosthesis in accordance with an embodiment of the present invention;

FIG. 22 is a cross sectional view of the prosthesis shown in FIG. 21 in a closed configuration;

FIG. 23 is a cross sectional view of the prosthesis shown in FIG. 21 in an open configuration;

FIG. 24A is a side view of an embodiment of a prosthesis in accordance with an embodiment of the present invention;

FIG. 24B is a partial sectional view of the embodiment shown in FIG. 24A;

FIG. 25 is a side view of an embodiment of a prosthesis in accordance with an embodiment of the present invention; and

FIG. 26 is a sectional view of an embodiment of a prosthesis positioned within the lower esophageal sphincter.

### DETAILED DESCRIPTION

The invention is described with reference to the drawings in which like elements are referred to by like numerals. The relationship and functioning of the various elements of this invention are better understood by the following detailed description. However, the embodiments of this invention are not limited to the embodiments illustrated in the drawings. It should be understood that the drawings are not to scale, and in certain instances details have been omitted which are not necessary for an understanding of the present invention, such as conventional fabrication and assembly.

As used in the specification, the terms proximal and distal should be understood as being in the terms of a physician delivering the valve to a patient. Hence the term “distal” means the portion of the valve that is farthest from the physician and the term “proximal” means the portion of the valve that is nearest to the physician.

The present invention relates to medical devices, and in particular to valves for implantation in a body lumen such as the lower esophageal sphincter or a vessel, preferably a vascular vessel. For example, the valves of the present invention are suitable for implantation into the vessels of the vasculature, such as veins, for regulating fluid flow through the vessel. As used herein, the term “implantable” refers to an ability of a medical device to be positioned at a location within a body, such as within a body lumen, either temporarily, semi-permanently, or permanently. Permanent fixation of the valve device in a particular position is not required. Furthermore, the terms “implantation” and “implanted” refer to the positioning of a medical device at a location within a body, such as within a body lumen.

The valves described herein are configured to be two-way valves. The valves are normally maintained in a closed configuration. The valves are configured to open in response to antegrade flow at a second pressure, for example from food or drink being swallowed by the patient and in response to retrograde flow at a third pressure, for example from vomiting or belching,

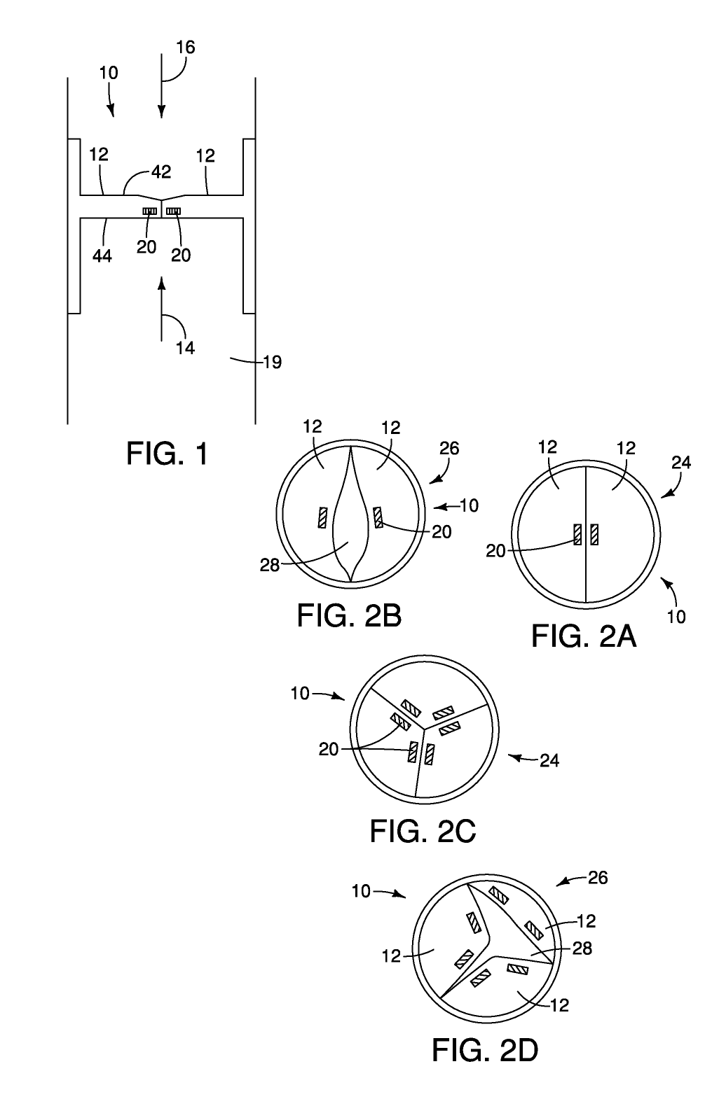

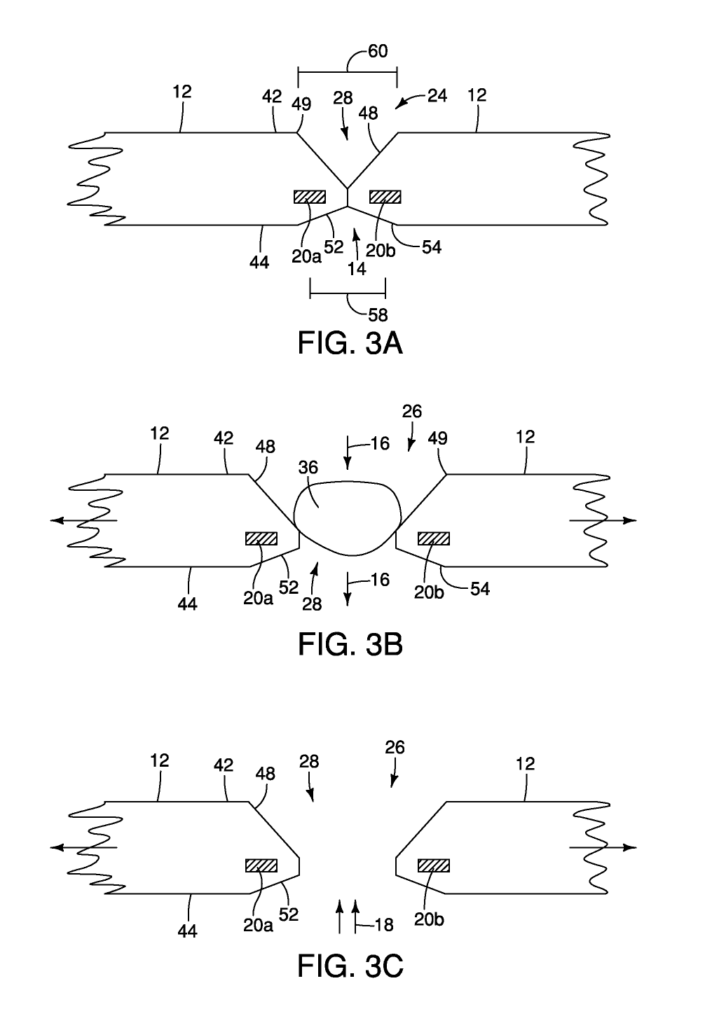

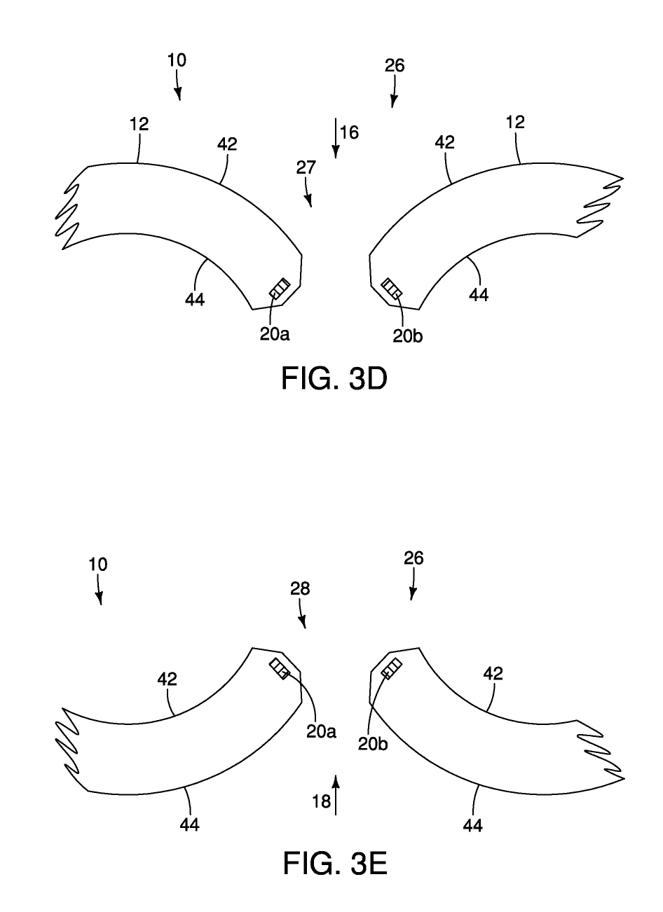

FIG. 1 illustrates a valve 10 in accordance with an embodiment of the present invention. The valve 10 includes at least one movable member 12 that is normally closed. A first pressure 14, for example a pressure that is normally present in the gastrointestinal tract, may be present and the valve 10 remains dosed. The movable member 12 is movable in response to a second pressure 16 and a third pressure 18 within a bodily lumen 19 of a patient, the third pressure 18 being greater than the second pressure 16 and both the second and third pressures 16, 18 being greater than the first pressure 14 (See FIGS. 3A-3E). By way of non-limiting example, the movable member 12 may be a leaflet or a sleeve. The valve 10 further includes at least one magnet 20. The valve 10 may include a first magnet 20a and a second magnet 20b having opposite poles that are attracted to each other to facilitate maintaining the valve 10 in a dosed configuration 24 shown in FIG. 2A where flow through the valve 10 is substantially prevented. The magnets 20 may be positioned at or near a valve lumen 28 formed in the valve 10 when the valve 10 is in an open configuration 26. In some embodiments, the magnets 20 may be positioned within the movable member 12, on a surface of the movable member 12 or may be a magnetic coating on the movable member 12.

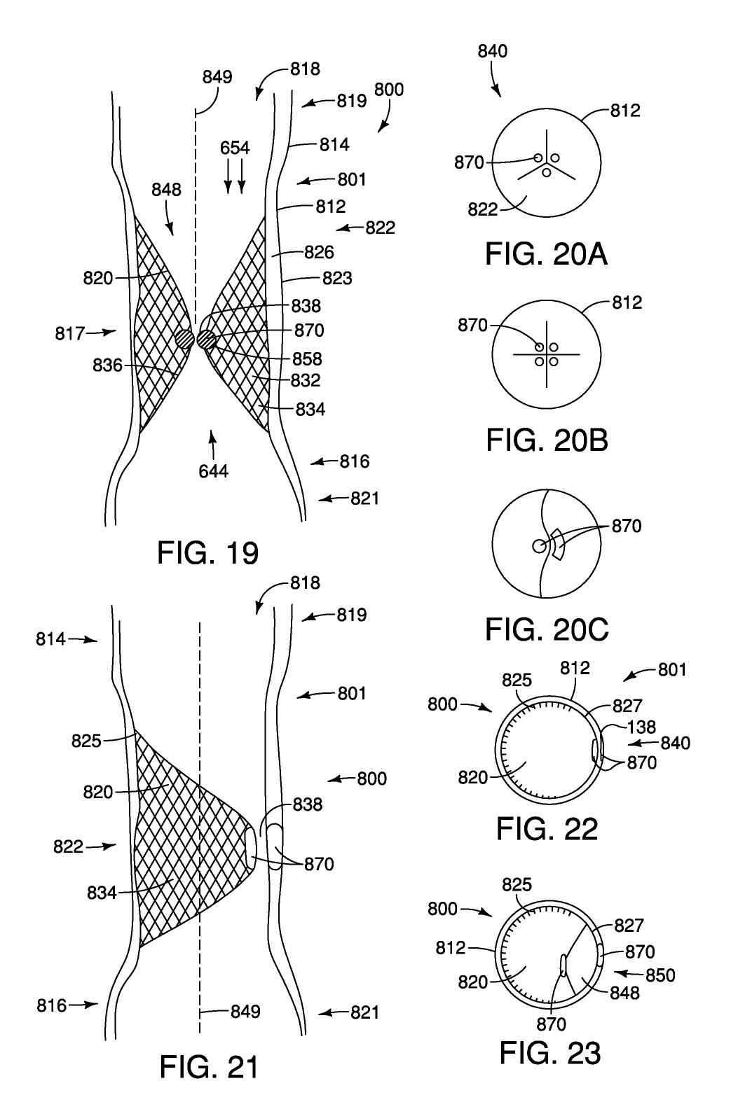

As shown in FIGS. 2A and 2B, some embodiments of the valve 10 may include two movable members provided as leaflets 12. FIG. 2B illustrates the valve 10 in the open configuration 26 where the magnets 20 are spaced apart relative to the closed configuration 24 and the valve lumen 28 is formed between the leaflets 12 so that fluids, solids and air may flow therethrough, The leaflets 12 coapt in the dosed configuration 24 and the magnets 20 facilitate maintaining the valve 10 in the dosed configuration, The first pressure 14 is not sufficient to move the leaflets 12 to the open configuration, for example the normal pressure that exists within the gastrointestinal tract. In some embodiments, the valve 10 may include one, three, four or more leaflets 12 that coapt to form the dosed configuration 24. FIGS. 20 and 2D illustrate the valve 10 having three leaflets 12 and a plurality of magnets 20. Other configurations are also possible for the magnets 20 and depend on the number of leaflets and the type of magnet.

FIGS. 3A-3E illustrate an embodiment of the valve 10 and the movable member 12 configurations in response to different pressures that are exerted on the valve 10. FIG. 3A shows the valve 10 in the dosed configuration 24. In the dosed configuration 24, the magnets 20a, 20b are attracted to each other and the movable members 12 coapt so that the lumen 28 of the valve 10 is substantially closed. Depending on the location of the valve 10, there may be a small amount of the first pressure 14 that is normally present, such as within the gastrointestinal tract where the first pressure 14 is not sufficient to interfere with the magnetic attraction of magnets 20a, 20b to move the valve 10 to the open configuration 26. The valve 10 is configured to prevent unintended retrograde flow through the valve lumen 28, such as reflux or aspiration of stomach contents especially when the patient is in a prone position.

FIG. 3B illustrates the valve 10 in the open configuration 26 in response the second pressure 16. The second pressure 16 may be exerted upon a first surface 42 of the valve 10 for example, from nutrients 36 including food and liquid that are passing from the mouth to the stomach through the valve 10. The second pressure 16 is sufficient to break the magnetic attraction between the magnets 20a, 20b and to move the movable members 12 so that the valve lumen 28 is in the open configuration 26 for the nutrients 36 to pass through. Once the nutrients 36 pass through the valve lumen 28, the magnetic attraction between magnets 20a, 20b encourages the movable members 12 to coapt and to reclose the valve lumen 28 so that the valve 10 returns to the dosed configuration 24 shown in FIG. 3A. The size and shape of the valve and the size, shape, length, materials, number, magnetic characteristics of the magnets can be tailored to provide the correct sealing strength to hold the valve 10 in the closed configuration 24 in the presence of the first pressure 14 and to allow the valve 10 to open in response to the second and third pressures 16, 18. The movable members 12 may also have varies shapes, lengths, materials, and numbers and may also include nitinol or other flexible support materials.

On occasion, the third pressure 18 may be exerted against a second surface 44 of the valve 10, for example from vomit or gas when the valve 10 is positioned in the gastrointestinal tract. As shown in FIG. 3C, the third pressure is sufficient to break the magnetic attraction between the magnets 20a, 20b and to move the movable members 12 so that the valve 10 is in the open configuration 26 and the valve lumen 28 is open to relieve the pressure and discomfort to the patient from the vomit or the gas. Once the third pressure 18 is relieved, the magnetic attraction between magnets 20a, 20b encourages the movable members 12 to coapt and to reclose the valve lumen 28 so that the valve 10 returns to the closed configuration 24 shown in FIG. 3A.

In some embodiments, the valve 10 may be shaped to facilitate the valve 10 moving between the open and closed configurations 24, 26 in response to the second and third pressures 16, 18. The shape of the valve 10 may be used to help control the amount of pressure needed to move the valve 10 to the open configuration 26. As shown in FIGS. 3A-3C, the first surface 42 includes a first recessed portion 48 that may be tapered toward the lumen opening 28. As shown in FIGS. 3A-3C, the first recessed portion 48 may be v-shaped with the widest portion 49 of the V of the recessed portion 48 at the first surface 42 and narrowing toward the lumen opening 28 of the valve 10. In other embodiments, the first recessed portion 48 may be curvilinear or angularly shaped and having the widest portion 49 of the recessed portion 48 at the first surface 42 and tapering inward to the lumen opening 28. The second pressure 16 is directed toward the movable members 12 where the magnets 20a, 20b are positioned at or near the lumen opening 28 to break the magnetic attraction between the magnets 20a, 20b and to move the valve 10 to the open configuration 26.

In some embodiments, the second surface 44 may not include a recess and may have a constant surface as shown in FIG. 1. As shown in FIG. 3A-3C, the second surface 44 may also include a second recessed portion 52. The second recessed portion 52 may be V-shaped with the widest portion 54 of the V of the recessed portion 52 at the first surface 44 and tapering inward toward the lumen opening 28 of the valve 10. A width 58 of the second recessed portion 52 at the widest portion 54 in the second surface 44 of the valve 10 in some embodiments may be narrower than a width 60 of the first recessed portion 48 at the widest portion 49 in the first surface 42. In some embodiments, the depth of the first recessed portion 48 may be greater than the depth of the second recessed portion 52. An overall surface area of the first recessed portion 48 may be greater than an overall surface area of the second recessed portion 52 so that the third pressure 18 against the second surface 44 that is required to open the valve 10 is greater than the second pressure 16 against the first surface 42. In other embodiments, the recessed portions 48, 52 may be curvilinear or angularly shaped and having a greater surface area in the first recessed portion 48 than the second recessed portion 52. Similar to the direction of the pressure described above, the third pressure 18 is directed toward the movable members 12 where the magnets 20a, 20b are positioned at or near the lumen opening 28 to break the magnetic attraction between the magnets 20a, 20b and to move the valve 10 to the open configuration 26. In some embodiments, a magnetic polymer or charged/electrostatic material may be provided in or on the movable members 12 instead of magnets.

FIGS. 3D and 3E illustrate the valve 10 in the open configuration 26 in response the second pressure 16 (FIG. 3D) and the third pressure 18 (FIG. 3E). The movable members 12 of the valve 10 may be made of a flexible material so that the movable members 12 bend in response to the pressures 16, 18 to open the valve 10. Similar to the embodiments described above, the magnets 20a, 20b facilitate the return of the valve 10 to the closed configuration (FIG. 3A) in the absence of the second and/or third pressure 16, 18.

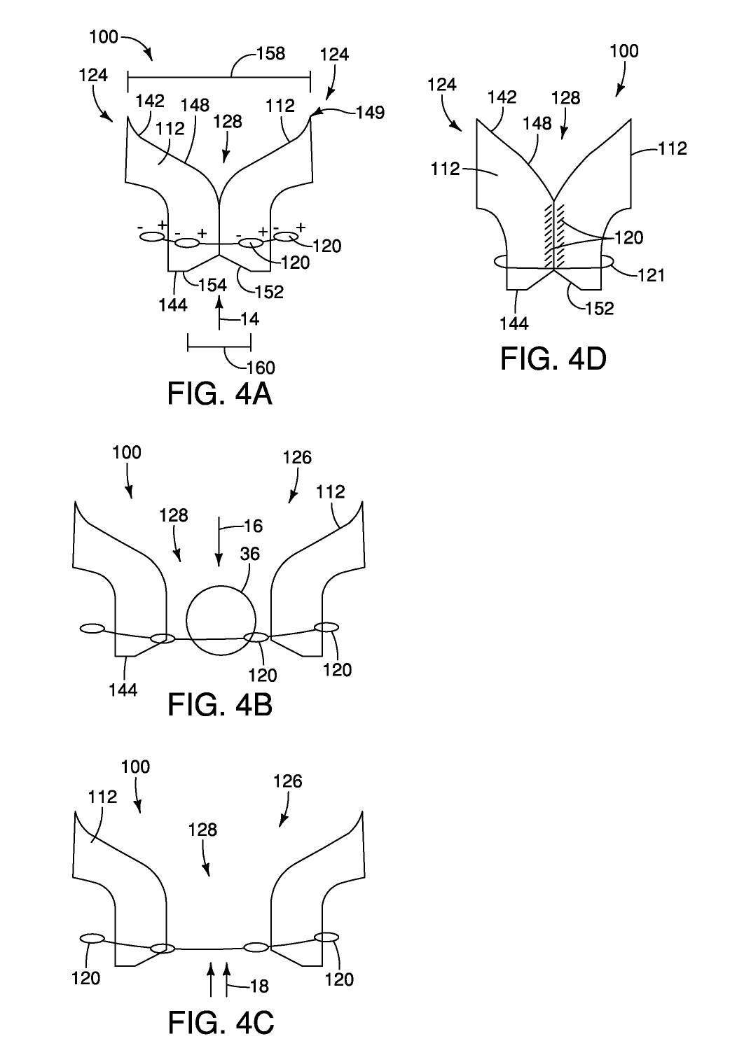

FIGS. 4A-4C illustrate an embodiment of the valve 100 and the leaflet 112 configurations in response to different pressures that are exerted on the valve 100 similar to the embodiment shown in FIGS. 3A-3C above. As shown in FIG. 4A, the valve 100 includes a plurality of movable members 112 and a plurality of magnets 120. The magnets 120 are positioned external to the movable members 112 and encircle the movable members 112. The magnets 120 may be connected with an elastomeric material 121 that expands and contracts as the valve 100 moves from a dosed configuration 124 to an open configuration 126. The material 121 may have spring-like closing effect on the valve 100, for example when the material 121 is an elastic band that springs back to the dosed configuration 124 once the second or third pressure 16, 18 is removed. In addition, the movable members 112 may have a spring-like dosing effect on the valve 100. The magnets 120 may include a positive and negative pole on each end that is attached to the opposite pole on the adjacent 120. FIG. 4A shows the valve 100 in the dosed configuration 124. In the dosed configuration 124, the magnets 120 are attracted to each other and the movable members 112 coapt so that a lumen 128 of the valve 100 is substantially dosed. Depending on the location of the valve 100, there may be a small amount of the first pressure 14 that is normally present, such as within the gastrointestinal tract where the first pressure 14 is not sufficient to interfere with the magnetic attraction of magnets 120 and the material 121 to move the valve 100 to the open configuration 126, The valve 100 is configured to prevent unintended retrograde flow through the valve lumen 128, such as reflux or aspiration of stomach contents especially when the patient is in a prone position.

FIG. 4B illustrates the valve 100 in the open configuration 126 in response the second pressure 16. The second pressure 16 may be exerted upon a first surface 142 of the valve 10 for example, from nutrients 36 including food and liquid that are passing from the mouth to the stomach through the valve 100. The second pressure 16 is sufficient to expand the elastomeric material 121 and to break the magnetic attraction between the magnets 120 to move the movable members 112 so that the valve lumen 128 is in the open configuration 126 for the nutrients 36 to pass through. Once the nutrients 36 pass through the valve lumen 128, the elastomeric material 121 and the magnetic attraction between magnets 120 encourages the movable members 112 to reclose the valve lumen 128 so that the valve 100 returns to the dosed configuration 124 shown in FIG. 4A,

On occasion, the third pressure 18 may be exerted against a second surface 144 of the valve 100, for example from vomit or gas when the valve 100 is positioned in the gastrointestinal tract. As shown in FIG. 40, the third pressure 18 is sufficient to expand the elastomeric material 121 and to interfere with the magnetic attraction between the magnets 120 and to move the movable members 112 so that the valve 100 is in the open configuration 126 and the valve lumen 128 is open to relieve the pressure and discomfort to the patient from the vomit or the gas. Once the third pressure 18 is relieved, the elastomeric material 121 and the magnetic attraction between magnets 120 encourages the movable members 112 to reclose the valve lumen 128 so that the valve 100 returns to the closed configuration 124 shown in FIG. 4A.

The valve 100 is similar to the valve 10 described above in that the valve 100 includes a first recessed portion 148 in the first surface 142 and a second recessed portion 152 in a second surface 144. As shown in FIGS. 4A-4C, the valve 100 includes a curvilinearly shaped first recessed portion 148 to facilitate the valve 100 moving between the open and dosed configurations 124, 126 in response to the second pressure 16. The second pressure 16 is directed toward the movable members 112 and into the first recessed portion 148 where the magnets 120 are positioned around the movable members 112 near the lumen opening 128 to break the magnetic attraction between the magnets 120 and to move the valve 100 to the open configuration 126. When the first pressure 16 is removed, the magnets 120 facilitate moving the valve 100 to the dosed configuration 124 shown in FIG. 4A.

As shown in FIG. 4A-4C, the second surface 144 includes the second recessed portion 152. The second recessed portion 152 may be v-shaped or may be curvilinear or angularly shaped similar to the portion 52 described above. A width 158 of the first recessed portion 148 at a widest portion 149 of the portion 148 is greater than a width 160 of the second recessed portion 152 at the widest portion 154, An overall surface area of the first recessed portion 148 is greater than an overall surface area of the second recessed portion 152 so that the third pressure 18 against the second surface 144 that is required to open the valve 100 is greater than the second pressure 16 against the first surface 142. In other embodiments, the recessed portions 148, 152 may be curvilinear or angularly shaped and having a greater surface area in the first recessed portion 148 than the second recessed portion 152. Similar to the direction of the pressure described above, the third pressure 18 is directed toward the movable members 112 where the magnets 120 are positioned at or near the lumen opening 128 to break the magnetic attraction between the magnets 120 and to move the valve 100 to the open configuration 126.

FIG. 4D illustrates an embodiment of the valve 100 in a dosed configuration 124 having the magnetic portion provided as a magnetic polymer, or charged/electrostatic material 120 provided on or in the movable members 112. The valve 100 shown in FIG. 4D functions like the embodiments described in FIGS. 4A-4C except that the magnetic portion 120 is provided in or on the movable members 112 instead of external to the movable members 112. The elastomeric material 121 is provided to facilitate return of the valve 100 to the dosed configuration 124, The elastomeric material 121 expands when the second and third pressures 16, 18 are present and contracts in the absence of the second and third pressures 16, 18.

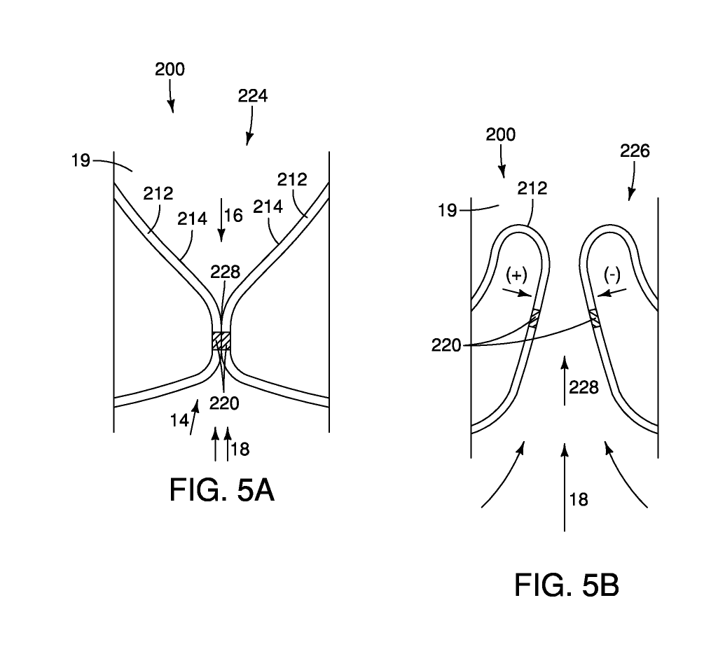

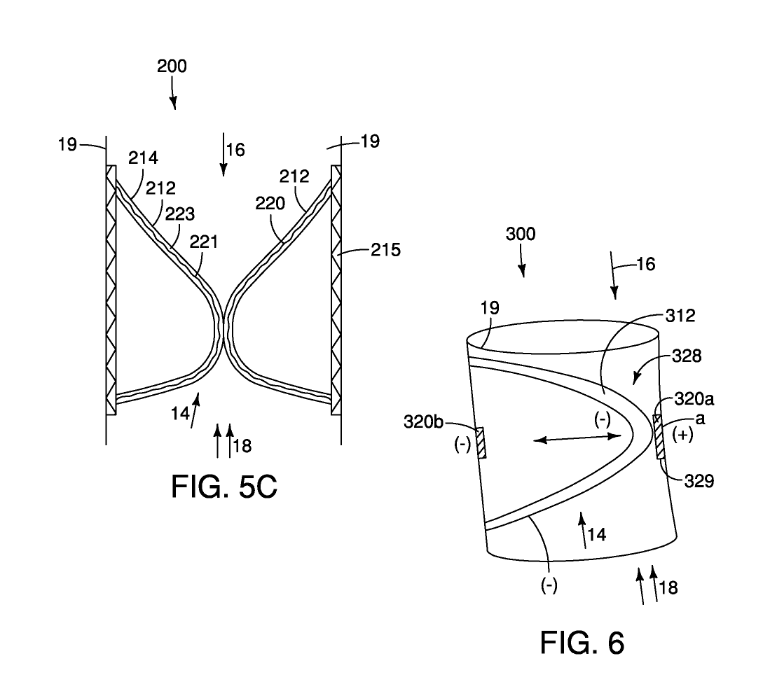

FIGS. 5A-5C illustrate an embodiment of a magnetic valve 200 including movable members 212 and magnets 220. The magnetic valve 200 is similar to the valves described above and responds to the second and third pressures 16 and 18 similarly. The movable members 212 of the valve 200 may be formed from a membrane 214 that attaches to the bodily lumen 19 as shown in FIG. 5A, The valve 200 may also include the magnets 220 positioned on or within the membranes 214 so that the valve 200 may be held in a dosed configuration 224 so that a lumen 228 of the valve 200 is substantially dosed. The magnets 220 may be asymmetrically positioned on or within the membranes 214 as shown in FIG. 5A, for example by distally positioning the magnets 220, The asymmetric positioning of the magnets 220 helps to facilitate the opening of the valve 200 in response to the different pressures 16, 18 in the different directions, FIG. 5B. illustrates the valve 200 in the open configuration 226 in response to the third pressure 18. As shown, the movable member 212 may be flexible so that the movable members 212 flex in the direction of the pressure 18 against the valve 200 and the movable members 212 move apart so that the lumen 228 is open. The magnets 220 are spaced apart in response to the third pressure. In the absence of the third pressure 18, the valve 200 returns to the dosed configuration 224 shown in FIG. 5A. In some embodiments, the moveable members may include nitinol or other flexible support material.

In some embodiments, the valve 200 may include an attachment body 215, such as a support or stent, to hold the valve 200 in position as shown in FIG. 5C. The attachment body 215 may be an expandable stent or a non-expandable stent. By way of non-limiting example, the stent may be an expandable or non-expandable stent. In some embodiments, the stent may be a self-expanding stent, such as a woven mesh formed from a metal or polymer or a laser cut pattern formed in a metal stent. The stent may also be formed from a bioabsorbable material. One example of a woven stent is the EVOLUTION® stent (Cook Medical, Inc., Bloomington, Ind.). Another exemplary stent may be a metal stent of the Gianturco type as described in U.S. Pat. No. 4,580,568. Other types of stents known to one skilled in the art may also be used. In some embodiments, the attachment body 215 may be a plurality of anchors that implant into the lumen wall of the patient to hold the valve 200 in position. The embodiment shown in FIG. 5C includes magnets 220 provided as a magnetic coating or a magnetic/electrostatic material 221 in or on the movable members 212. The magnetic coating or material 221 may be provided over an entire luminal face 223 of the movable member 221 or a portion thereof. In some embodiments, the magnetic coating or material may be provided as a magnetic foil or magnetic polymer, Any of the embodiments described herein may include the magnetic coating, magnetic/electrostatic material or magnets to facilitate closing of the valves and controlled opening in response to different pressures.

FIG. 6 illustrates an embodiment of a magnetic valve 300. The valve 300 includes a movable member 312 and magnets 320a and 320b having opposite charges. In the embodiment shown, the movable member 312 may be formed from or coated with a magnetic foil or magnetic polymer having a negative charge. The movable member 312 may be sized to extend across the bodily lumen 19 so a valve lumen 328 is formed away from a center of the valve 300 and positioned along a wall 329 of the valve 300. The wall 329 may be provided as a stent, support or a frame that facilitates implantation of the valve 300 within the bodily lumen. The magnet 320a may be positively charged and may be positioned at or near the valve lumen 328 so that the movable member 312 is normally attracted to the magnet 320a and the valve 300 is in a closed configuration 324 as shown in FIG. 6. The magnetic attraction between the magnet 320a and the movable member 312 is sufficient to maintain the valve 300 in the dosed configuration 324 in the presence of the first pressure 14. In response to the second pressure 16 or the third pressure 18, the movable member 312 is moved away from the magnet 320a so that the lumen opening 328 is formed and fluid or air can pass therethough with the valve 300 in an open configuration (not shown). The magnet 320b may have a negative charge so that in the absence of the second pressure 16 or the third pressure 18, the magnet 320b repels the movable member 312 and the movable member is moved back into contact with the magnet 320a so that the valve 300 is maintained in the dosed configuration 324.

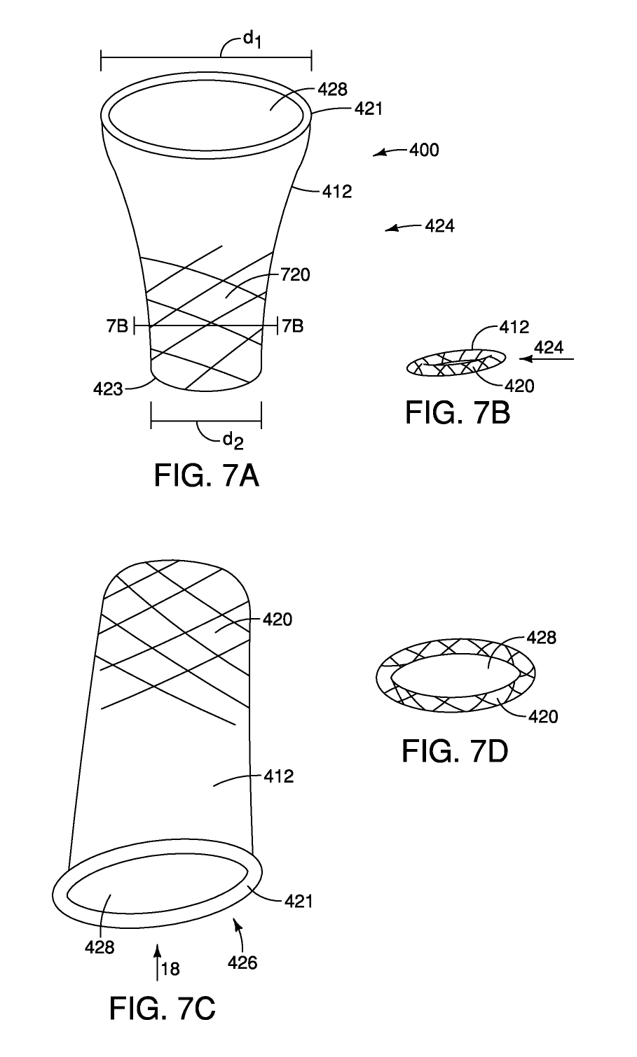

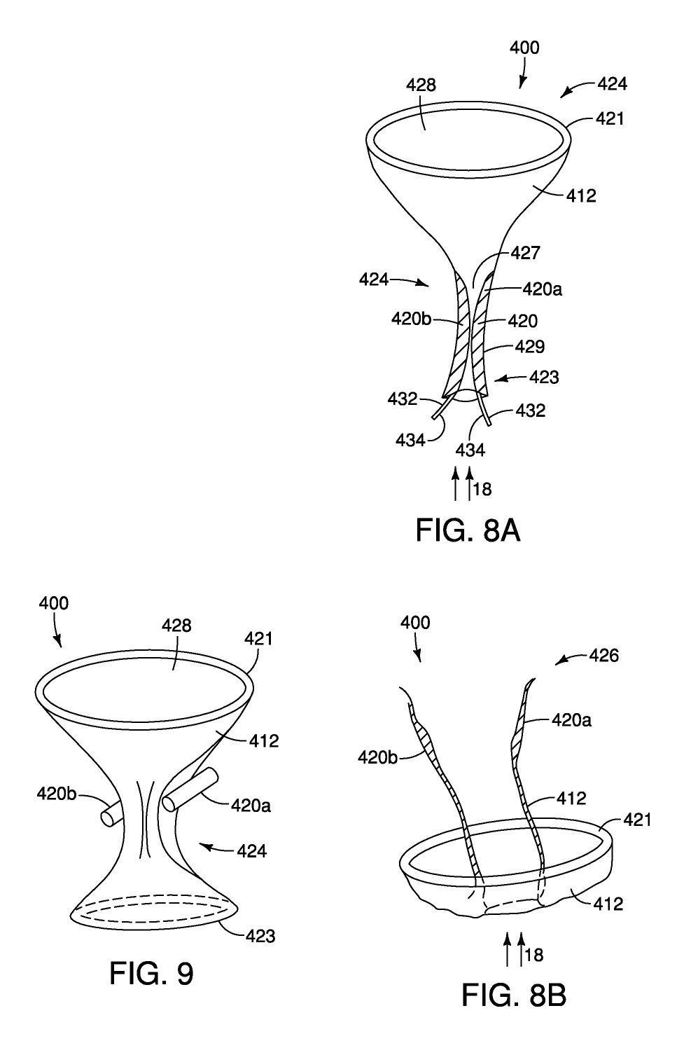

FIGS. 7A-9 illustrate embodiments of a magnetic valve 400 including a movable member 412 and a magnetic portion 420. The magnetic valve 400 is similar to the valves described above and responds to the second and third pressures 16 and 18 similarly. A dosed configuration 424 is shown in FIGS. 7A, 8A and 9. A lumen 428 is formed through the valve 400 in response to the second and third pressures 16, 18 similar to the embodiments described above. The open configuration 426 is shown in FIGS. 7C and 8B with the movable member 412 everted in response to the third pressure 18. The magnetic valve 400 is normally maintained in the closed configuration 424 until sufficient pressure against the magnetic portion 420 breaks the magnetic connection and the lumen 428 is opened. In the embodiments of the magnetic valve 400 illustrated in FIGS. 7A-9, the movable member 412 is shown as a sleeve. The sleeve 412 includes a proximal portion 421 and a distal portion 423. In some embodiments, the proximal portion 421 has a larger diameter d₁ than a diameter d₂ of the distal portion 423. The sleeve may include a magnetic portion 420 that is configured to control the dosing force needed in the absence of the second and third pressures 16, 18 to keep the valve 400 in the dosed configuration 424. In some embodiments, a very thin sleeve 412 may be used with the magnetic portion 420 controlling the sealing of the valve.

As shown in FIGS. 7A-7D, the magnetic portion 420 may be provided as a magnetic coating or a magnetic foil on the distal portion 423 of the sleeve 412. A cross-sectional view through line 7-7 is shown in FIG. 7B where the valve 400 is in the closed configuration 424 in the absence of pressure or in the presence of the first pressure 14. The open configuration 426 is shown in FIGS. 7C and 7D where the sleeve 412 is everted on response to the third pressure 18 and the lumen 428 is open so that retrograde flow can pass through the lumen 428.

As shown in FIG. 8A, the magnetic portion 420 may be provided as magnets 420a, 420b connected to the sleeve 412, either on an inner surface 427 or an outer surface 429 that have a magnetic attractive force that maintains the valve 400 in the closed configuration 424 in the absence of pressure or in the presence of the first pressure. The magnets 420a, 420b may also be imbedded within the sleeve 412. The sleeve 412 may also include distal flaps 432 extending from the distal portion 423 of the sleeve 412. The distal flaps 432 may be configured to provide a surface 434 against which the third pressure may exert force to facilitate opening of the valve 400. FIG. 8B illustrates the valve 400 in the open configuration 426 with the sleeve 412 being everted in response to the third pressure. Retrograde flow can pass through the lumen 428 with the valve 400 in the open configuration. The embodiment of the valve 400 shown in FIG. 9, illustrates a pair of magnets 420a, 420b positioned external to the sleeve 412, The magnets 420a, 420b may be rod-shaped as shown, although any shape magnet may be used. The magnets 420a, 420b may extend across a portion of the sleeve 412 to dose the lumen 428 of the valve 400.

The embodiments of the valve 400 shown in FIGS. 7A-9 may also include a support, attachment or stent as discussed above. In some embodiments, the valve 400 may be connected directly to the luminal wall of the patient without a support, attachment or stent.

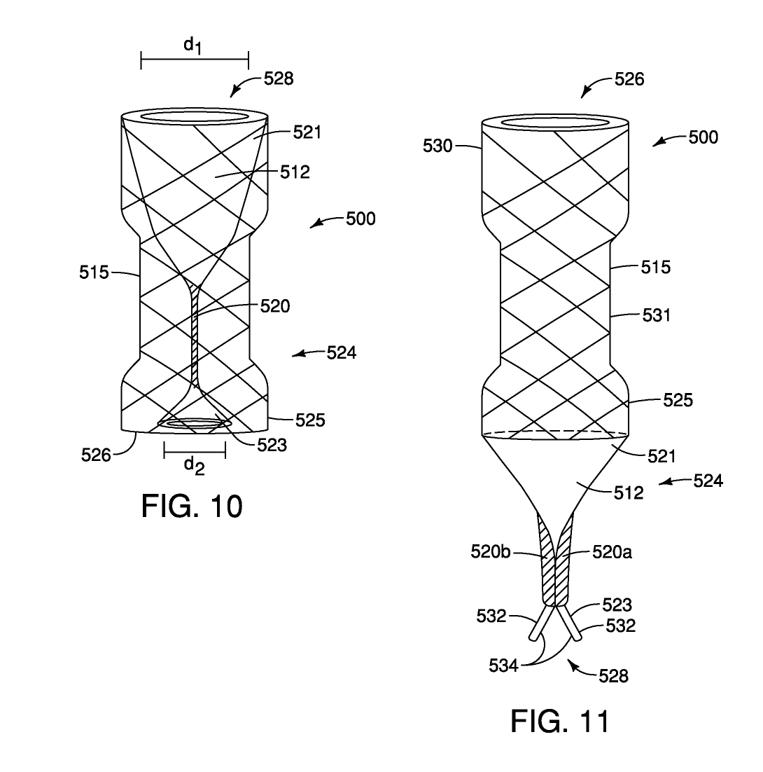

FIGS. 10 and 11 embodiments of a magnetic valve 500 including a movable member 512 and a magnetic portion 520. The valve 500 may be provided with a stent 515. The magnetic valve 500 is similar to the valves described above and responds to the first, second and third pressures 14, 16 and 18 similarly. A dosed configuration 524 is shown and a lumen 528 is formed through the valve 500 in response to the second and third pressures 16, 18 similar to the embodiments described above. The magnetic valve 500 is normally maintained in the closed configuration 524 until sufficient pressure against the magnetic portion 520 breaks the magnetic connection and the lumen 528 is opened. In the embodiments of the magnetic valve 500 illustrated in FIGS. 10 and 11, the movable member 512 is shown as a sleeve. The sleeve 512 includes a proximal portion 521 and a distal portion 523. In some embodiments, the proximal portion 521 has a larger diameter d₁ than a diameter d₂ of the distal portion 523.

As shown in FIG. 10, the sleeve 512 may be positioned internal to the stent 515 so that the distal portion 523 is proximal to a distal end 525 of the stent 515. In some embodiments, the distal portion 523 of the sleeve 512 may extend distal to the stent 515 and have the magnetic portion 520 positioned within a lumen 526 of the stent 515. The magnetic portion 520 may be provided as a magnetic foil, a magnetic polymer or a pair of magnets as described in the embodiments above.

As shown in FIG. 11, the sleeve 512 may extend distal to the distal end 525 of the stent 515. The stent 515 may be coated or the sleeve 512 may extend to a proximal end 530 of the stent 515 so that fluids and other particulate matter does not pass through a wall 531 of the stent 515 and instead moves through the lumen 526 of the stent 515. The lumen 528 of the sleeve 512 may be connected to the lumen 526 of the stent 515, for example when the sleeve 512 extends distal to the distal end 525 of the stent 515. The lumen 528 of the sleeve 512 may also extend through the stent 515 from the proximal end 530 of the stent 515.

As shown in FIG. 11, the magnetic portion 520 may be provided as magnets 520a, 520b connected to the sleeve 512 and have a magnetic attractive force that maintains the valve 500 in the closed configuration 524 in the absence of pressure or in the presence of the first pressure 16. The magnets may be similar to the embodiments described above. The sleeve 512 may also include distal flaps 532 extending from the distal portion 523 of the sleeve 512. The distal flaps 532 may be configured to provide a surface 534 against which the third pressure 18 may exert force to facilitate opening of the valve 500.

The embodiments described above are suitable for placement within a lower esophageal sphincter or in any other lumens of the gastrointestinal tract. Some embodiments are also suitable for positioning within the vascular system. By way of non-limiting example, the embodiments may be positioned within a vein to replace or supplement a defective venous valve to allow flow in an antegrade direction and to substantially prohibit flow in a retrograde direction.

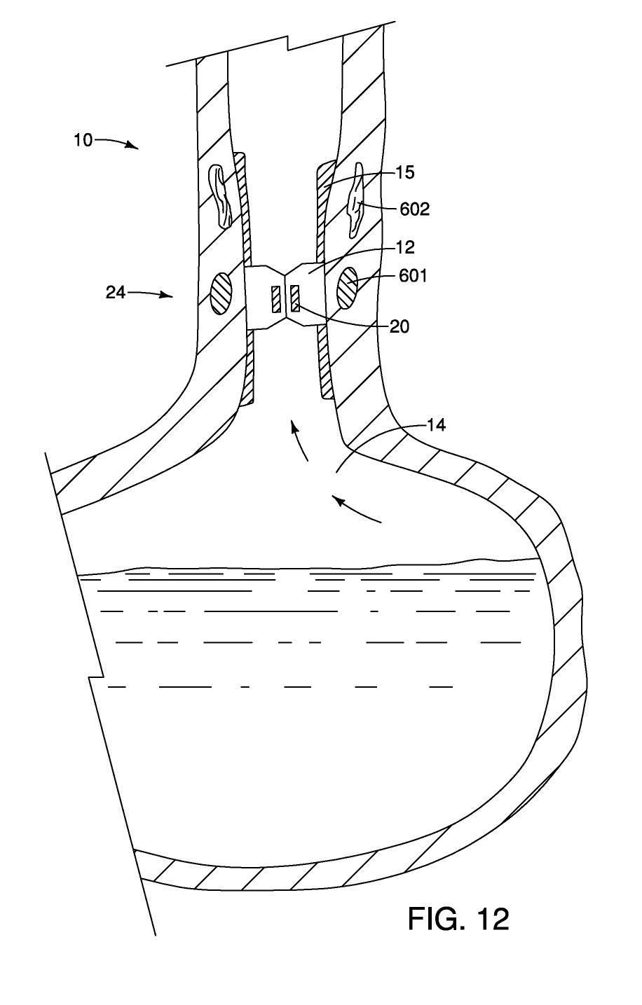

An embodiment of the magnetic valve 10 is shown positioned in the lower esophageal sphincter (LES) 601 and cancerous tumor 602. As shown in FIG. 12, the valve 10 includes a stent 15, movable members 12 and magnets 20. The magnetic valve 10 is shown in the closed configuration 24 in response to the first pressure 14. However, any liquid or food 36 is readily passed in an antegrade direction through the esophageal stent and into the stomach. (See for example FIG. 3B.) As a result, magnetic valve 10 opens to provide flow in the antegrade direction. Conversely, any fluids or food 36 are prevented from flowing into the retrograde direction due to the magnetic closure of the valve 10. However, when the pressure of the gas or fluid in the stomach builds so as to cause the patient to belch or vomit and produce the third pressure 18, the magnet valve 10 will open. (See for example FIG. 3C.)

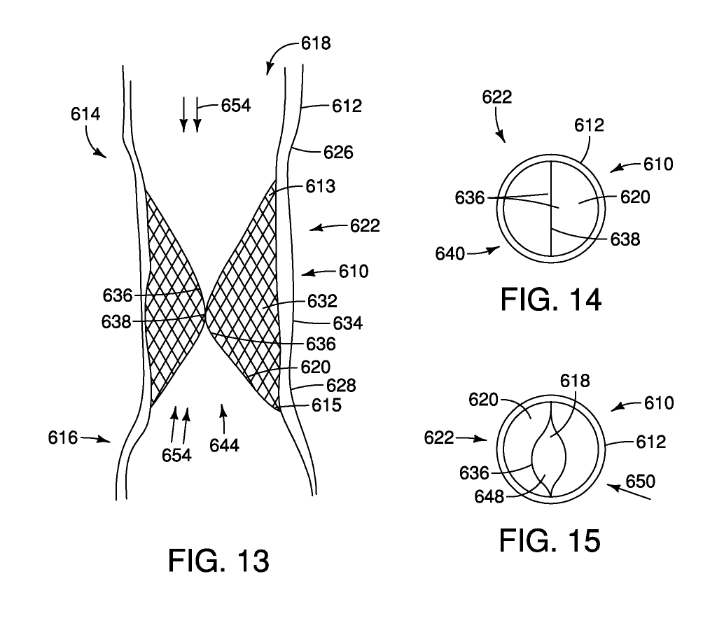

FIG. 13 illustrates a prosthesis 610 in accordance with an embodiment of the present invention. As shown in FIG. 13, the prosthesis is provided as a stent 610 that includes a tubular body 612 having a proximal portion 614, a distal portion 616 and a lumen 618 extending therethrough. The stent 610 may be an expandable or non-expandable stent. In some embodiments, the stent 610 may be a self-expanding stent, such as a woven mesh formed from a metal or polymer or a laser cut pattern formed in a metal stent. The stent may also be formed from a bioabsorbable material. One example of a woven stent is the EVOLUTION® stent (Cook Medical, Bloomington, Ind.). Another exemplary stent may be a metal stent of the Gianturco type as described in U.S. Pat. No. 4,580,568. In some embodiments, the stent may be a non-expandable tubular stent formed from a polymer. Other types of stents known to one skilled in the art may also be used.

As shown in FIG. 13, the stent 610 further includes a membrane 620 positioned within the lumen 618 of the tubular body 612 that forms a valve 622 for controlling flow through the stent 610. The membrane 620 may be connected to the body 612 so that a proximal portion 613 of the membrane 620 is connected to the proximal portion 614 of the body 612 and a distal portion 615 of the membrane 620 is connected to the distal portion 616 of the body 612. In some embodiments, the body 612 may include a coating 626 that forms a liquid barrier between the lumen 618 and an exterior 628 of the body 612. In some embodiments, the coating 626 may be an elastomer such as silicone or polyurethane but the coating 626 is not limited to elastomers. In some embodiments, for example with an uncoated stent, having a woven body 612, the membrane 620 may be used to form a barrier between the lumen 618 and the exterior 628 of the body 612. The coating and/or the membrane may be connected to the stent by any method, including but not limited to dipping, spraying, gluing, ultrasonic welding, RF welding and laser welding.

A chamber 632 is formed between the membrane 620 and the coating 26 or within the membrane 620 itself. The chamber 632 may be inflated with a filling substance 634 so that at least a portion 636 of the membrane 620 extends into the lumen 618 and occludes at least a portion of the lumen 618. A contact region 638 is formed where the portion 636 extends into the lumen 618. The membrane 620 is connected to the body 612 so that fluid and particulate flow occurs through the lumen 618 and not between the membrane 620 and the body 612. As shown in FIG. 13, the membrane 620 may protrude into the lumen 618 so that an hourglass shaped passageway is formed.

As illustrated in FIG. 14, the valve 622 positioned within the tubular body 612 is normally in a dosed position 640 so that the lumen 618 is dosed to fluid and particulate flow therethrough by the membrane 620 at the contact region 638. The valve 622 remains closed with a first pressure 644 being exerted against the valve 622. The first pressure 644 may be the pressure that is normally present within the gastrointestinal tract, by way of non-limiting example. The valve 622 is configured to prevent unintended flow through the valve lumen 618, such as reflux or aspiration of stomach contents especially when the patient is in a prone position. In the dosed position 640, the portions 636 of the membrane 620 extending into the lumen 618 are joined at the contact region 638 so that fluid and other substances do not pass through the lumen 618.

FIG. 15 illustrates the valve 622 in an open configuration 650 with an opening 648 formed in the lumen 618 of the body 612. The valve 622 is configured to open in response to a second pressure 654 that is greater than the first pressure 644. In some embodiments, the second pressure 654 may be the result of peristaltic motion that lengthens and opens the valve 622. In embodiments where the stent 610 is positioned in the LES, the second pressure 654 may be from liquids or particulate matter ingested by the patient and that flows in the antegrade direction or the second pressure 654 may be due to belching or vomiting that flows in the retrograde direction through the valve 622.

In the embodiment of the stent 610 shown in FIG. 13, the membrane 620 may be filled with the filling substance 634 so that the portion 636 of the membrane 620 extends into the lumen 618 of the body 612, The filling substance 634 may be compressible or the membrane 620 may be flexible or both so that in the presence of the second pressure 654, the filling substance 634 and/or the membrane 620 are moved so that the opening 648 is formed. When the second pressure 654 is removed, the valve 622 returns to the dosed configuration 640. In some embodiments, a pressure within the chamber 632 is greater than the first pressure 644 outside the chamber 632 so that the portions 636 form a closed valve 622. The force required to compress the filling substance 634 is greater than the force of the first pressure 644 so the valve remains in the dosed configuration 640 in response to the first pressure 644. The force required to compress the filling substance 634 is less than the second pressure 654 so that the valve 622 is moved to the open configuration 650 in response to the second pressure 654.

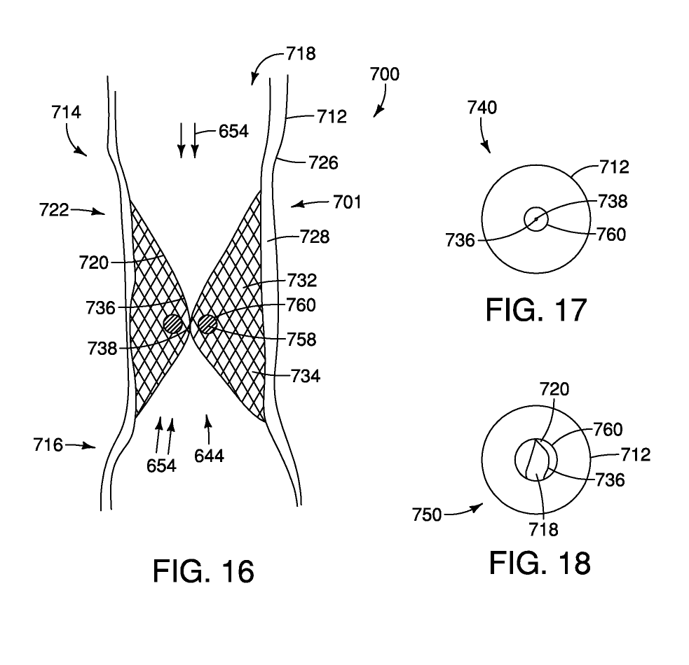

FIG. 16 illustrates an alternative embodiment of a prosthesis 700 in accordance with the present invention. The prosthesis 700 includes a tubular body 712 having a proximal portion 714, a distal portion 716 and a lumen 718 extending therethrough. The prosthesis 700 may be provided a stent 701 that may be a self-expandable or a balloon expandable stent similar to the embodiments described above.

As shown in FIG. 16, the stent 701 further includes a membrane 720 positioned within the lumen 118 of the tubular body 712 that forms a valve 722 for controlling flow through the stent 701, The membrane 720 may be connected to the body 712 at the proximal portion 714 and the distal portion 716. In some embodiments, the body 712 may include a coating 726 that forms a liquid barrier between the lumen 718 and an exterior 728 of the body 712. In some embodiments, for example with an uncoated stent 701 having a woven body 712, the membrane 720 may be used to form a barrier between the lumen 718 and the exterior 728 of the body 712. The membrane 720 is connected to the body 712 so that no fluid flows between the membrane 720 and the body 712. A chamber 732 is formed between the membrane 720 and the coating 726 or within the membrane 720 itself. The chamber 732 may be inflated with a filling substance 734 so that at least a portion 736 of the membrane 720 extends into the lumen 718 and occludes at least a portion of the lumen 718. A contact region 138 is formed where the portion 736 extends into the lumen 718.

As shown in FIG. 16, the valve 722 further includes a closure member 758 in the form of an elastomeric ring 760. The elastomeric ring 760 may be used to facilitate closure of the lumen 718 in response to the first pressure 644 so that the valve 722 is normally in a closed configuration 740 shown in FIG. 17, The first pressure 644 may be the pressure that is normally present within the gastrointestinal tract, by way of non-limiting example. The valve 722 is configured to prevent unintended flow through the valve lumen 718, such as reflux or aspiration of stomach contents especially when the patient is in a prone position. In the dosed position 740, the portions 736 of the membrane 720 extending into the lumen 718 are joined at the contact region 738 and the elastomeric ring 760 extends through the membrane 720 to facilitate closure so that fluid and other substances do not pass through the lumen 718. The elastomeric ring 760 is expandable so that in response to the second pressure 654, the valve 722 may be moved to an open configuration 750 shown in FIG. 18.

FIG. 19 illustrates an alternative embodiment of a prosthesis 800 provided as a stent 801 in accordance with the present invention, The stent 801 includes a tubular body 812 having a proximal portion 814, a distal portion 816 and a lumen 818 extending therethrough. The stent 801 may be a self-expandable or a balloon expandable stent similar to the embodiments described above. The stent 801 may be coated or non-coated as described above, In some embodiments, the stent 801 may include a proximal end 819 and a distal end 821 where the proximal end 819 or the distal end 821 or both have an enlarged diameter relative to a diameter of a central portion 823 of the body 812. The enlarged diameter proximal and distal ends of the stent may also be provided with other embodiments described herein.

As shown in FIG. 19, the stent 801 further includes a membrane 820 positioned within the lumen 818 of the tubular body 812 that forms a valve 822 for controlling flow through the stent 801. The membrane 820 may be connected to the body 812 at the proximal portion 814 and the distal portion 816 or to a central portion 817. The membrane 820 is connected to the body 812 so that no fluid flows between the membrane 820 and the body 812. A chamber 832 is formed between the membrane 820 and a stent coating 826 or within the membrane 820 itself, The chamber 832 may be inflated with a filling substance 834 so that at least a portion 836 of the membrane 820 extends into the lumen 818 and occludes at least a portion of the lumen 818. A contact region 838 is formed where the portion 836 extends into the lumen 818.

As shown in FIG. 19, the valve 822 further includes a closure member 858 provided in the form of magnets 870. The magnets 870 may be used to facilitate closure of the lumen 818 in response to the first pressure 644 so that the valve 822 is normally in a closed configuration 840 shown in FIGS. 20A-20C. The valve 822 responds to the first pressure 644 and the second pressure 654 similar to the embodiments described above. In the closed position 840, the portions 836 of the membrane 820 extending into the lumen 818 are joined at the contact region 838 and the magnets 870 are attracted to each other to facilitate closure so that fluid and other substances do not pass through the lumen 818. The magnets 870 may be provided in a plurality of different numbers and configurations as illustrated by examples shown in FIGS. 20A-20C. By way of non-limiting example, 2, 3, 4 or more magnets 870 may be provided. In some embodiments, the magnets 870 may be formed to have complimentary shapes as shown in FIG. 200. The magnets 870 may be positioned within the membrane 820, partially within the membrane 820 or external to the membrane 820 such as a magnetic coating. The second pressure 654 is sufficient to break the magnetic attraction between the magnets 870 to move the valve 822 to an open configuration 850 (see FIG. 23) and form an opening 848. In some embodiments, the opening 848 is formed along a central axis 849 through the valve 822. In other embodiments, the opening 848 may be offset from the central axis 849. When the second pressure 654 is removed, the magnets 870 facilitate the return of the valve 822 to the closed configuration 240.

FIG. 21 illustrates an alternative embodiment of the prosthesis 800 showing the valve 822 extending across the lumen 818 so that when the valve 822 is in the open configuration 850 shown in FIG. 23, the opening 848 is offset from the central axis 849 through the lumen. The prosthesis 800 further includes the membrane 820 positioned within the lumen 818 of the tubular body 812. The membrane 820 may be connected to a portion 825 of the body 812 while a second portion 827 of the body 812 is free from connection with the membrane 820. This configuration allows the membrane 820 to move away from the second portion 827 of the body 812 to move the valve 822 to the open configuration 850 shown in FIG. 23 in response to the second pressure 654. The prosthesis 800 is normally in the dosed configuration 840 shown in FIG. 22. As shown in FIGS. 21-23, the prosthesis 800 includes a plurality of magnets 870. in some embodiments, one magnet 870 may be positioned within, partially within or external to the membrane 820 and another magnet 870 may be positioned on the body 812 so that the membrane 820 extends across the lumen 818 in the closed configuration 840 shown in FIG. 22. The magnets 870 join at the body 812. Other features of the embodiment shown in FIG. 21 are similar to the embodiments described above.

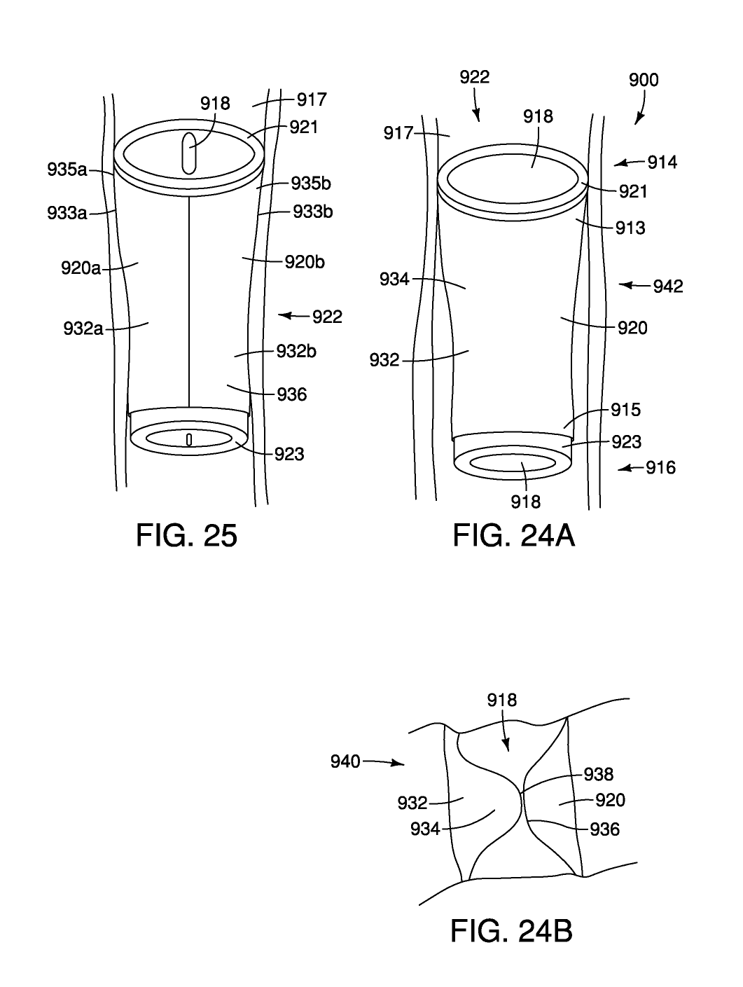

FIGS. 24A, 24B and 25 illustrate a prosthesis 900 in accordance with an embodiment of the present invention. As shown in FIG. 24A, the prosthesis 900 includes a tubular body 912 extending from a proximal portion 914 to a distal portion 916 and a lumen 918 extending therethrough. A proximal support structure 921 of the tubular body 912 is connected to a body lumen 917 and is configured to provide a fluid tight connection so that any fluid flowing through the body lumen 917 is directed through the prosthesis 900. The proximal support structure 921 may be expandable or non-expandable. The prosthesis 900 also includes a membrane 920 operably connected to the proximal support structure 921 and to a distal support structure 923. The distal support structure 923 may be free from connection to a wall of the body lumen 917. A proximal portion 913 of the membrane 920 is connected to the proximal support structure 921 and a distal portion 915 of the membrane 920 is connected to the distal support structure 923. The membrane 920 forms a valve 922 for controlling flow through the prosthesis 900.

A chamber 932 is formed in the membrane 920 by the membrane 920 folding on itself and forming the chamber 932 therebetween. In some embodiments, the membrane 920 is sealed at the proximal portion 913 against the proximal support structure 921 and at the distal portion 915 against the distal support structure 923. The chamber 932 may be inflated with a filling substance 934 so that at least a portion 936 of the membrane 920 extends into the lumen 918 and occludes at least a portion of the lumen 918. A contact region 938 is formed where the portion 936 extends into the lumen 918 as shown in the sectional view in FIG. 24B.

The valve 922 is normally in a dosed position 940 so that the lumen 918 is dosed to fluid and particulate flow therethrough by the membrane 920 at the contact region 938 similar to the embodiments described above. The valve 922 remains dosed with a first pressure 644 being exerted against the valve 922. The first pressure 644 may be the pressure that is normally present within the gastrointestinal tract, by way of non-limiting example. The valve 922 is configured to prevent unintended flow through the valve lumen 918, such as reflux or aspiration of stomach contents especially when the patient is in a prone position. In the dosed position 940, the portions 936 of the membrane 920 extending into the lumen 918 are joined at the contact region 938 so that fluid and other substances do not pass through the lumen 918. The valve 922 may be moved to an open configuration 950 in response to the second pressure 654 that is greater than the first pressure as described above.

FIG. 25 illustrates an embodiment of the prosthesis 900 that is similar to the embodiment shown in FIGS. 24A and 24B. In addition, the prosthesis shown in FIG. 25 includes a first membrane 920a and a second membrane 920b that extend between the proximal support structure 921 and the distal support structure 923 to form the valve 922. Both the proximal and distal support structures 921, 923 may be secured to a wall of the body lumen 917. A sealed chamber 932a in the first membrane 920a and a sealed chamber 932b in the second membrane 920b may be formed by sealing the first and second membranes around a perimeter 933a, 933b of the membranes 920a, 920b to the body lumen 917 and to the proximal and distal support structures 921, 923. An opening 935a, 935b may be left in the perimeter 933a, 933b so that the chambers 932a, 932b may be filled with the filling substance 936 after the prosthesis is positioned in the body lumen 917. In some embodiments, the chamber 932a, 932b may be formed by folding the membrane 920a, 920b over on itself as described above. The prosthesis 900 operates similarly to the embodiments described above in response to the first and second pressures 644, 654.

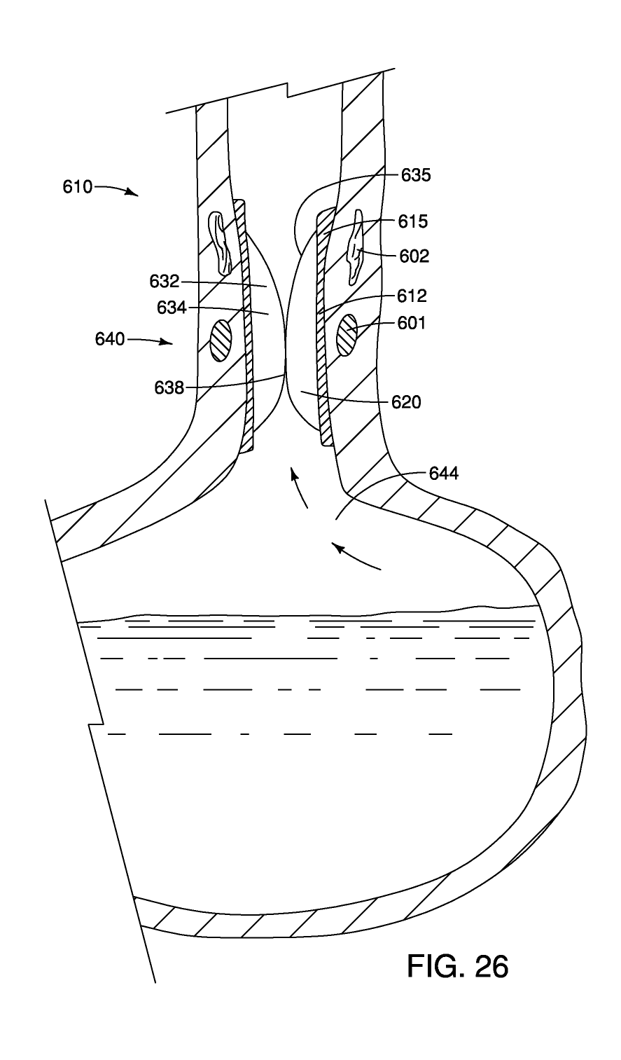

FIG. 26 illustrates an embodiment of the stent 610 positioned in the lower esophageal sphincter (LES) 601 and cancerous tumor 602. As shown, the stent 610 includes the body 612 with the membrane 620 forming the chamber 632. The chamber 632 is filled with the filling substance 634 so that the contact portion 638 forms the dosed configuration 640 in response to the first pressure 644. However, the stent 610 is movable to the open configuration 650 in response to the second pressure 654 (not shown).

In some embodiments, the chamber 632 may be filled with the filling substance 634 prior to delivering the stent 610 to the LES 601. In some embodiments, an opening 635 in the membrane 620 may be left unjoined to provide an aperture for injecting the filling substance into the chamber 632 for inflation of the membrane 620 during placement of the stent 610 in a desired bodily location. Filling during positioning of the stent 610 may allow for individual differences in the size of the placement site and the desired pressure within the membrane 620 to allow the dosed configuration 640 in response to the first pressure 44 and the open configuration 650 in response to the second pressure. The opening 635 may be sealed after placement of the stent 610.

Suitable inflating filling substances include, but are not limited to, any biocompatible materials that are movable to open the stent to the open configuration in response to the second pressure. Non-limiting examples of inflating media may include gases, solids, liquids, gels, or foams, such as a collagenous fill material, a remodelable or absorbable material, a biocompatible polymer, an aqueous buffer such as saline, a non-resorbable material, ECM rods or particulates, a collagenous or gelatinous foam, air, chitosan, gelatin, oxidized regenerated cellulose, calcium alginate, alginate, thrombin-fibrin enhanced materials, fibrin glues, or any suitable combination thereof.

The embodiments described above are suitable for placement within a lower esophageal sphincter or in any other lumens of the gastrointestinal tract. Some embodiments are also suitable for positioning within the vascular system. By way of non-limiting example, the embodiments may be positioned within a vein to replace or supplement a defective venous valve to allow flow in an antegrade direction and to substantially prohibit flow in a retrograde direction.

The above Figures and disclosure are intended to be illustrative and not exhaustive. This description will suggest many variations and alternatives to one of ordinary skill in the art. AH such variations and alternatives are intended to be encompassed within the scope of the attached claims. Those familiar with the art may recognize other equivalents to the specific embodiments described herein which equivalents are also intended to be encompassed by the attached claims.

1. A medical device for controlling flow through a bodily lumen, the medical device comprising:
a valve device positioned within the bodily lumen to control the flow through the lumen, the valve device comprising:
a movable member sized and shaped to substantially close the bodily lumen in a first configuration, the movable member movable from the first configuration in the presence of a first pressure within the bodily lumen to a second configuration in response to a second pressure within the bodily lumen that is greater than the first pressure, wherein the valve device is open in the second configuration for flow therethrough, the movable member normally being positioned in the first configuration; and
a magnetic portion operably connected to the movable member to facilitate positioning the movable member in the first configuration, the magnetic portion having a magnetic attraction to another magnetic portion or a portion of the valve device having a charge opposite a charge of the magnetic portion.

2. The medical device of claim 1, wherein the movable member comprises a sleeve or a plurality of leaflets.

3. The medical device of claim 1, wherein the magnetic portion comprises a magnetic foil or a magnetic polymer.

4. The medical device of claim 1, wherein the magnetic portion is external to the movable member and operably surrounds the at least a portion movable member.

5. The medical device of claim 1, wherein the valve further comprises an elastomeric member external to and surrounding at least a portion of the moveable member.

6. The medical device of claim 1, wherein the movable member comprises a first surface on a proximal portion of the valve device and a second surface on a distal portion of the valve device, the first surface comprises a first recess exposed to the second pressure.

7. The medical device of claim 6, wherein the second surface comprises a second recess.

8. The medical device of claim 7, wherein the first surface recess comprises a greater area than an area of the second surface recess.

9. The medical device of claim 2, wherein the sleeve further comprises a surface against which a third pressure provides an opening force.

10. A medical device for controlling flow through a bodily lumen in a first direction and a second direction, the medical device comprising:
a tubular member having a proximal portion, a distal portion and a lumen therethrough; and
a valve device operably connected to the lumen to control the flow through the lumen in the first direction and the second direction, the valve device comprising:
a membrane having a proximal portion and a distal portion; the membrane operably connected to the tubular member, at least a portion of the membrane a forming a sealed chamber by sealing the proximal portion of the membrane to the proximal portion of the tubular member and sealing the distal portion of the membrane to the distal portion of the tubular member or by sealing the membrane to itself and forming the chamber between the proximal and distal portions of the membrane, the sealed chamber extending inward into the lumen and substantially dosing the lumen in a first configuration at a first pressure within the lumen, the membrane having a second configuration in response to a second pressure that is greater than the first pressure wherein the lumen is open in the second configuration for flow therethrough, the membrane normally being positioned in the first configuration, the sealed chamber comprising a filling substance;
wherein the tubular member, the filling substance or both are deformable in response to the second pressure.

11. The medical device of claim 10, further comprising a closure member to facilitate closing of the lumen in the first configuration.

12. The medical device of claim 11, wherein the closure member comprises a magnetic portion or an elastomeric ring.

13. The medical device of claim 10, further comprising a second membrane forming a second sealed chamber so that the membranes contact each other in the second configuration.

14. The medical device of claim 10, wherein the chamber defines an hourglass shaped passageway extending therethrough.

15. The medical device of claim 10, wherein the chamber has an annular configuration providing a central opening through the lumen in the second configuration and the proximal portion of the membrane is sealed around its perimeter to the proximal portion of the tubular member and the distal portion of the membrane is sealed around its perimeter to the distal portion of the tubular member.

16. The medical device of claim 10, wherein the chamber has an asymmetrical configuration providing an opening through the lumen in the second configuration, the opening offset from a central axis of the tubular member.

17. The medical device of claim 1, wherein the tubular member is an expandable stent or a support comprising a proximal end and a distal end connected by the membrane extending therebetween.

18. A method of controlling flow through a bodily lumen, the method comprising:
providing a valve device comprising a movable member and a magnetic portion;
positioning the valve device within the bodily lumen so that the valve is in a first configuration so that the bodily lumen is substantially dosed in to the presence of a first pressure and the magnetic portion facilitates positioning the movable member in the first configuration, the magnetic portion having a magnetic attraction to another magnetic portion or a portion of the valve device having a charge opposite a charge of the magnetic portion; and
moving the movable member to a second configuration in response to a second pressure within the bodily lumen that is greater than the first pressure so that the magnetic attraction is disrupted and opening the valve device in the second configuration for flow in a first direction therethrough.

19. The method of claim 18, further comprising wherein the movable member comprises a sleeve and providing a contact surface on the sleeve to facilitate moving the sleeve to the open configuration.

20. The method of claim 18, comprising returning the valve device to the first configuration after the second pressure is relieved by attraction of the magnetic portion to close an opening in the valve device.
